(19) United States
(12) Patent Application Publication
(10) Pub. No.: US 2025/0040685 A1
(43) Pub. Date: Feb. 6, 2025

(54) ROBOTIC STAMP FOR COSMETIC APPLICATOR CONFIGURED FOR USERS WITH LIMITED MOBILITY

(71) Applicant: L'OREAL, Paris (FR)

(72) Inventors: Gregoire CHARRAUD, Jersey City, NJ (US); Casey BARBARINO, San Anselmo, CA (US); Hyosik Dennis MIN, Palisades Park, NJ (US)

(73) Assignee: L'OREAL, Paris (FR)

(21) Appl. No.: 18/228,364

(22) Filed: Jul. 31, 2023

(51) Int. Cl. A45D 40/12; A45D 40/26

(52) U.S. Cl. CPC A45D 40/12; A45D 40/26; A45D 2040/0006; A45D 2200/10

(57) ABSTRACT

A stamp is provided that is configured to apply a cosmetic formulation to a user. The stamp includes a recessed area configured to receive the cosmetic formulation, the recessed area having a shape confirming to a target area of the user for applying the cosmetic formulation; a connector configured to attach to an external device; and processing circuitry configured to control an angular position of the stamp as the stamp is moved toward the target area of the user.

### FIELD

The present disclosure describes a system and features related to a device for modifying, mitigating, altering, reducing, compensation for, or the like, the movement of a cosmetic applicator caused by unintentional movements, tremors, limited mobility, or the like of a user.

### BACKGROUND

Unintentional movements of the human body, or human tremors, can occur in individuals suffering from motion disorders or even healthy individuals. Due to these unintentional movements, a person may have difficulty in performing a task that requires care and precision, such as applying a cosmetic composition to a part of the body, such as the face, hands, or feet.

Therefore, there is a need for a solution that allows application of a cosmetic composition that is compatible with the diverse and disposable nature of cosmetic applicators.

### SUMMARY

In an embodiment, a stamp device is provided to apply a cosmetic formulation to a user, comprising: a recessed area configured to receive the cosmetic formulation, the recessed area having a shape confirming to a target area of the user for applying the cosmetic formulation; a connector configured to attach to an external device; and processing circuitry configured to control an angular position of the stamp as the stamp is moved toward the target area of the user.

In an embodiment, the cosmetic formulation is lipstick and the recessed area has a shape confirming to a user's lip region.

In an embodiment, the stamp includes an embedded camera and proximity sensor that are configured to detect the target area of the user as the stamp is moved toward the target area of the user.

In an embodiment, the stamp includes a 2-axis gimbal motor system that is controlled by the processing circuitry to control the angular position of the stamp.

In an embodiment, the stamp includes a mounting mechanism configured to attach the stamp to a mobile user device.

In an embodiment, the stamp includes a connector configured to attach and communicatively couple the stamp to a motion stabilizing device.

In an embodiment, a system is provided that includes: a stamp configured to apply a cosmetic formulation to a user, the stamp including: a recessed area configured to receive the cosmetic formulation, the recessed area having a shape confirming to a target area of the user for applying the cosmetic formulation, and a connector configured to attach to a motion stabilizing device; and the motion stabilizing device configured to receive information of the target area of the user and control an angular position of the stamp as the stamp is moved toward the target area of the user.

### BRIEF DESCRIPTION OF THE DRAWINGS

A more complete appreciation of the embodiments and many of the attendant advantages thereof will be readily obtained as the same becomes better understood by reference to the following detailed description when considered in connection with the accompanying drawings, wherein:

FIG. 1 shows a motion stabilizing device.

FIG. 2 shows how the motion stabilizing device couples with an adaptor and a make-up applicator.

FIG. 3A shows a diagram of the internal components of motion stabilizing device.

FIG. 3B shows a diagram of an alternative embodiment of the motion stabilizing device in which the receiver portion includes an electromagnetic positioner instead of the motive elements shown in FIG. 3A.

FIG. 4 shows an overview of a universal adapter handle connection system.

FIGS. 5A-5E show different view of the stamp module for the lipstick application example.

FIG. 6 shows an embodiment where the robotic stamp is mounted to a smart stick.

FIG. 7 shows movement of the robotic stamp is controlled within the stamp itself.

FIG. 8 shows a system that utilizes the robotic stamp when it is mounted on the motion stabilizer.

FIG. 9 shows a method performed according to the system of FIG. 8.

FIGS. 10A and 10B shown an embodiment where the robotic stamp is mounted to a user's smartphone directly.

FIG. 11 shows a method performed according to the system of FIG. 10.

FIGS. 12A-12B show how the stamp can be made to be personalized for the user based on the user's morphology and drawing objective.

### DETAILED DESCRIPTION

The present disclosure describes a cosmetic applicator system that minimizes modifies, mitigates, alters, reduces, compensates for, or the like unintentional movements by stabilizing, orienting, operating, controlling, etc. an applicator for a user and is also designed to be flexible to accommodate different types of commercially available cosmetic applications. The present disclosure further describes a system and features to enhance the functionality of such a cosmetic applicator system.

The basic features and operation of a motion stabilizing device for a cosmetic applicator is described in U.S. Pat. No. 11,458,062, which is incorporated herein by reference.

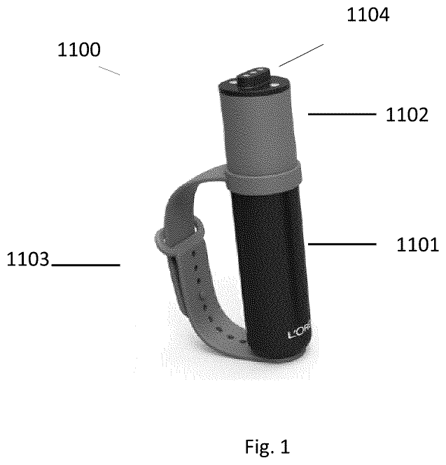

FIG. 1 shows a conventional motion stabilizing device 1100, which serves as a base unit for receiving a cosmetic applicator according to an embodiment. The device 1100 includes a handle portion 1101, a receiver portion 1102 and a strap 1103. The receiver portion 1102 includes an interface 1104, shown as a male connector that couples with a cosmetic applicator, which will be discussed in detail below. The receiver portion could be utilized for communication between the base unit and the applicator. The connection to an adaptor and/or an applicator could be accomplished with a mechanical coupling, such as screw-in or snap-fit, or it could be accomplished with magnets.

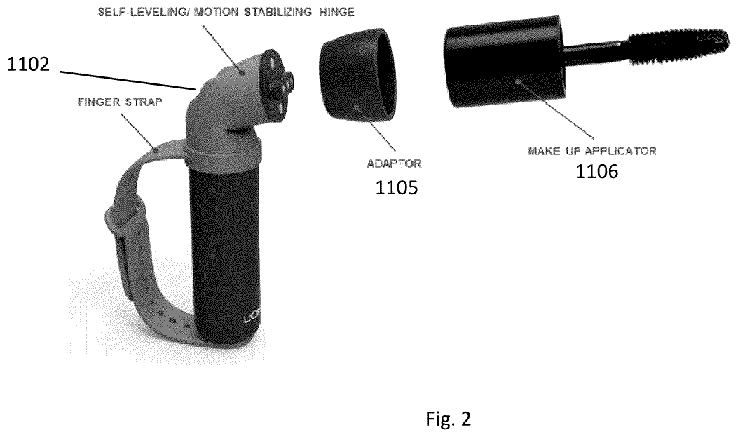

FIG. 2 shows how the device 1100 couples with an adaptor 1105 and a make-up applicator 1106. It can be seen that the adaptor fits over the exposed end of the receiver portion 1102. The adaptor includes electrical mating connectors (a female connector—not shown) in a recessed portion to make contact with the electric interface of the receiver portion 1101.

As shown in FIG. 2, the receiver portion 1102 is configured to contort, articulate, reposition, etc., between an upright posture (as shown in FIG. 1) and an angled posture (as shown in FIG. 2). This is accomplished with a hinge mechanism contained inside the receiver portion 1102. FIG. 2 shows that the hinge mechanism is a self-leveling/motion stabilizing hinge.

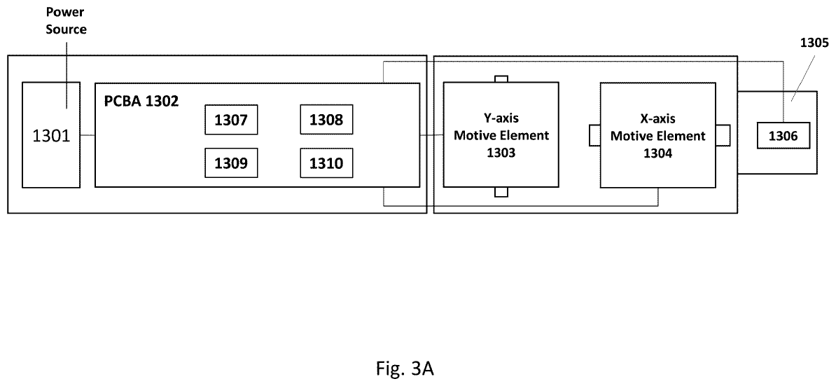

FIG. 3A shows a diagram of the internal components of device 1100 according to one embodiment. In the handle portion, the device includes a power source 1301, which may be a battery or the like. The device includes a printed circuit board assembly (PCBA) 1302, which may include positional sensor circuitry 1307, reader circuitry 1308, control circuitry 1309, and communication interface 1310, as understood in the art.

For instance, as the sensor circuitry 1307, the PCBA may include at least one inertial sensor and at least one distributed motion sensor to detect unintentional muscle movements and measure signals related to these unintentional muscle movements that are created when a user adversely affects motion of the applicator. These sensors also detect the motion of the stabilized output relative to device. The control circuitry sends voltage commands in response to the signals to the motion generating elements (described below) to cancel the user's tremors or unintentional muscle movements. This cancellation maintains and stabilizes a position of the applicator, keeping it stable.

One of ordinary skill in the art readily recognizes that a system and method in accordance with the present invention may utilize various implementations of the control circuitry and the sensor circuitry and that would be within the spirit and scope of the present invention. In one embodiment, the control circuitry 1309 comprises an electrical system capable of producing an electrical response from sensor inputs such as a programmable microcontroller or a field-programmable gate array (FPGA). In one embodiment, the control circuitry comprises an 8-bit ATMEGA8A programmable microcontroller manufactured by Atmel due to its overall low-cost, low-power consumption and ability to be utilized in high-volume applications.

In one embodiment, the at least one inertial sensor in the sensor circuitry is a sensor including but not limited to an accelerometer, gyroscope, or combination of the two. In one embodiment, the at least one distributed motion sensor in the sensor circuitry is a contactless position sensor including but not limited to a hall-effect magnetic sensor.

The system created by the combination of the sensor circuitry, the control circuitry, and the motion generating elements may be a closed-loop control system that senses motion and acceleration at various points in the system and feeds detailed information into a control algorithm that moves the motion-generating elements appropriately to cancel the net effect of a user's unintentional muscle movements and thus stabilize the position of the applicator. The operation and details of the elements of the control system and control algorithm are understood in the art, as described in U.S. PG Publication 2014/0052275A1, incorporated herein by reference.

The communication interface 1310 may include a network controller such as BCM43342 Wi-Fi, Frequency Modulation, and Bluetooth combo chip from Broadcom, for interfacing with a network.

In the receiver portion of the device, there may be two motive elements to allow 3-dimensional movement of the receiver as anti-shaking movement. The two motive elements include a y-axis motive element 1303 and an x-axis motive element 1304, each being connected to and controlled by the PCBA 1302. Each of the motive elements may be servo motors as understood in the art. The device further includes end effector coupling 1305, which is configured to couple with the adaptor 1105. The end effector coupling 1305 may include a radiofrequency identification (RFID) reader 1306, configured to read an RFID tag, which may be included with the applicator, as will be discussed below.

FIG. 3B shows a diagram of an alternative embodiment of the device 1100 in which the receiver portion includes an electromagnetic positioner 1311 instead of the motive elements shown in FIG. 3A. The electromagnetic positioner 1311 may include U-shaped magnetic cores 1312 arrayed around a non-magnetic tube 1313, which is filled with a magnetic fluid 1314. Each of the magnetic cores has arm portions that are surrounded by windings 1315. The magnetic cores may be controlled by the control circuitry in the PCBA 1302 to act as a controllable active magnetic field-generating structure which is used to generate a variable magnetic field that acts upon the magnetic fluid, causing it to be displaced, thereby enabling the armature to be moved to a desired coordinate position and/or orientation. The details of implementing the electromagnetic positioner 1311 may be found in U.S. Pat. No. 6,553,161, which is incorporated herein by reference.

In the above-described conventional motion stabilizing device, there is a problem that the interface 1104 that receives the adaptor 1105 requires a specific point of attachment to align properly with the interface.

Therefore, the below embodiments provide a universal adapter connection between the handle of the motion stabilizing device in order to improve user experience and reduce the struggle and time taken to set up the system for use.

In one embodiment, the present disclosure is directed towards a cosmetic applicator. The cosmetic applicator can be used for a variety of cosmetics and cosmetic applications, including, but not limited to, mascara, eyeliner, eyebrow products, lip products (lipstick, lip gloss, lip liner, etc.), skin products, and/or hair products. In one embodiment, the cosmetic applicator can include an adapter, wherein the adapter can connect the cosmetic applicator to a motion stabilizer. The motion stabilizer can be, for example, a handle that can counteract unintentional motions such as tremors or spasms. These motions can interfere with the application of cosmetics and can also make it difficult to generally interact with cosmetic applicators or tools. For example, the many cosmetic products require a twisting motion or force to be applied to open or extrude the product. It can be difficult for users to achieve the range of motion or the precision necessary to apply these forces to the cosmetic. In one embodiment, the cosmetic applicator can hold a cosmetic and can enable the proper force to be applied to the cosmetic to open, close, mix, stir, blend, extrude, or achieve other similar functions necessary for application.

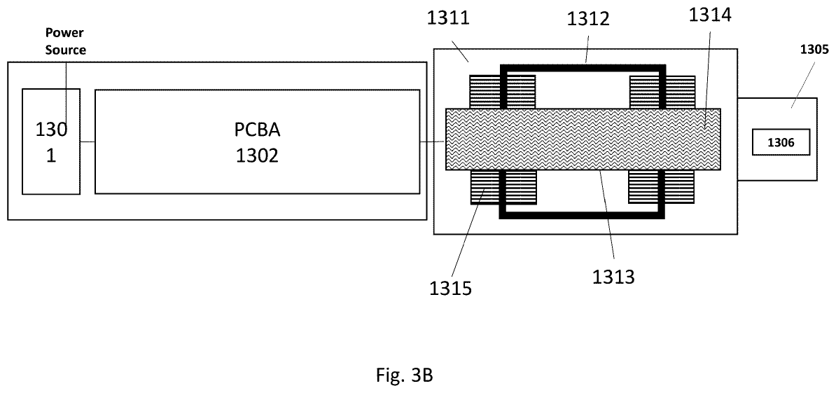

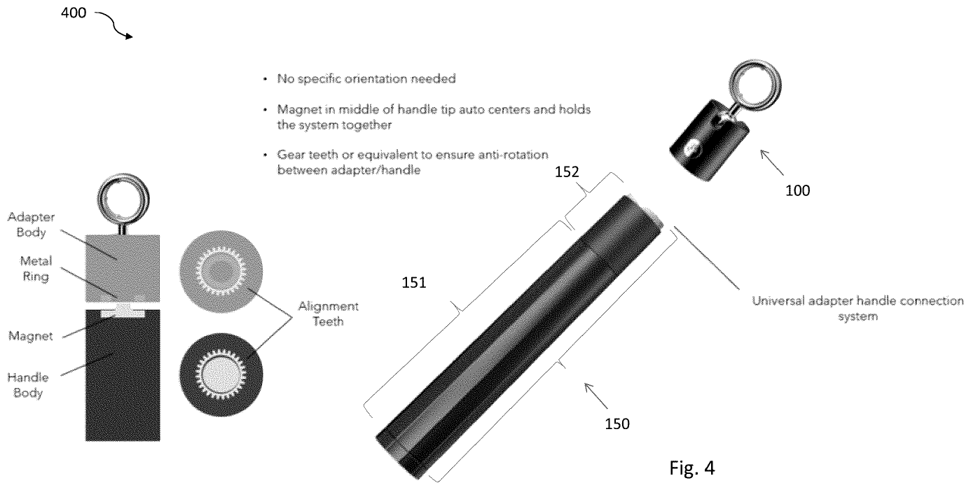

FIG. 4 shows an overview of a universal adapter handle connection system 400. The system includes a motion stabilizer device 150 that includes a handle portion 151 and a hinge portion 152 (receiver portion) that is functionally similar to the device 1100 shown in FIG. 1. It further includes a universal adapter 100 that attaches to the device 150 and also holds different types of cosmetic applicators. The basic features and operation of universal adapter handle connection system 400 are described in co-pending U.S. application Ser. Nos. 18/091,882; 18/091,920; 18/091,843; 18/091.925; 18/148,957; 18/148,880; and Ser. No. 18/148,930, which are incorporated herein by reference.

The above-described embodiments describe a ring-shaped adapter for holding a cosmetic applicator in which a gimble system can be used to stabilize or improve the user's gesture. However, the drawing parts still requires precision skill and time. Therefore, the below embodiments describe a robotic “stamp” that will facilitate the application of certain types of make-up. With the stamp, instead of stabilizing the regular make up applicator, controlling the position of the stamp and making sure the transfer is on target will increase the quality of the pattern and reduce the speed of application.

It will be seen in the below description, that when using such a stamp, an objective becomes achieving a position controlled stamp that adapts an angle and position function of user alignment with the face/body part targeted.

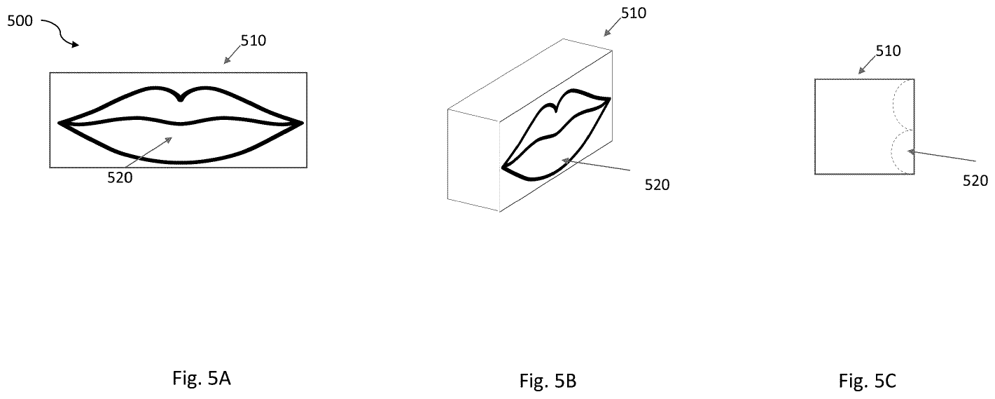

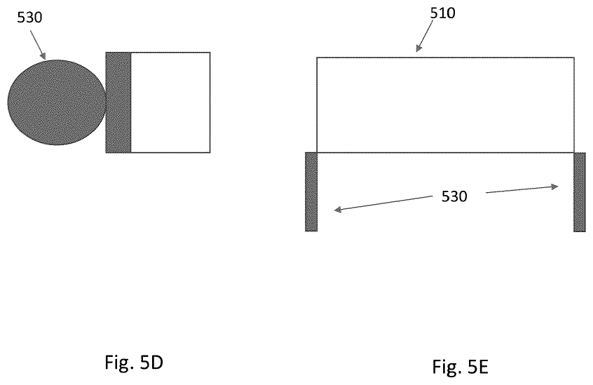

FIGS. 5A-5E show different view of the stamp module 500 for the lipstick application example. FIG. 5A shows a front view that includes a stamp body 510 and a lip-shaped recess portion 520. As will be described later, the stamp shape is personalized based on the user's morphology and the drawing objective. The material of the stamp can be one of many materials that will receive lipstick and allow it be re-transferred to the user's lips with ease. Such materials may be rubber, silicon, foam, spongy material, or plastic. Alternatively, the stamp itself may be made of the based lipstick material itself in the manufacturing process.

FIG. 5B shows a perspective view of the stamp body 510. FIG. 5C shows a side view with a transparent perspective, where the depth of the lip-shaped recess portion is illustrated.

FIG. 5D shows a side view, where each side has a clip 530 for potentially attaching to a smart phone, which will be described in a later embodiment.

FIG. 5E shows an overhead view that also includes a view of the stamp body 500 and the clips 520.

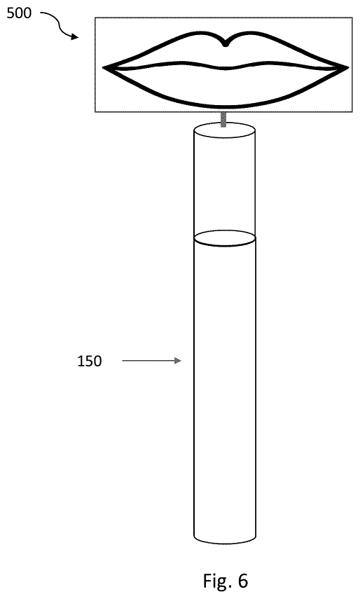

FIG. 6 shows an embodiment where the robotic stamp 500 is mounted to a smart stick. The smart stick may be the above-describe motion stabilizer 150 and the stamp may utilize a magnet ring similar the magnet ring shown in FIG. 4 for the universal adapter.

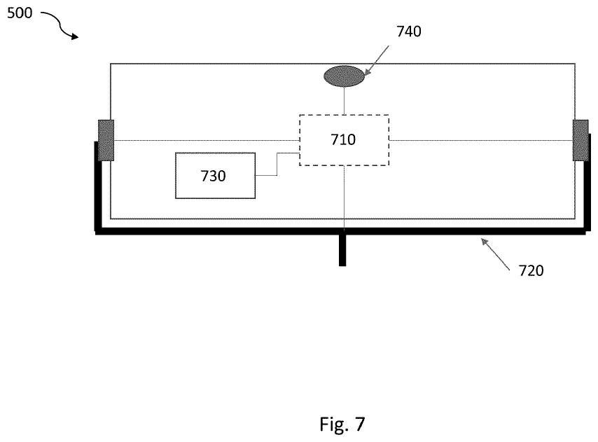

FIG. 7 shows movement of the robotic stamp is controlled within the stamp itself. Stamp includes a microprocessor 710 that is connected to a 2-axis gimbal motor system 720. The microprocessor is further connected to a power source 730, which may be a “button battery” as known in the art. In an alternative embodiment, the adapter may receive power from the stabilizer device 150 through the receiver interface of the stabilizer device. The stamp may further include an embedded camera system and proximity sensor 740, which can be used to better ensure robotic control while the user is attempting to bring the stamp to the desired area.

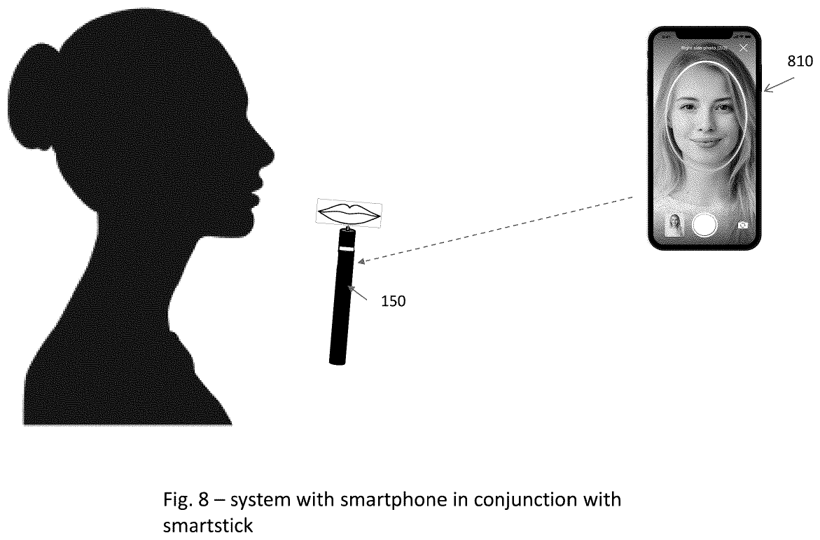

FIG. 8 shows a system 800 that utilizes the robotic stamp 500 when it is mounted on the motion stabilizer 150. The system further includes a smartphone 810. The camera on the smartphone may be used to capture an area of interest on the user's face, such the user's lips in this example. The area of interest may be transmitted wirelessly to the motion stabilizer 150. Then, while the user is attempting to apply the stamp their lips, the embedded camera system and proximity sensor to ensure robotic control while the user is bringing the stamp to the desired area. With the area of interest being provided by the smartphone, the embedded camera can focus on the proper target, the device can adjust the angular position until the skin is contacted.

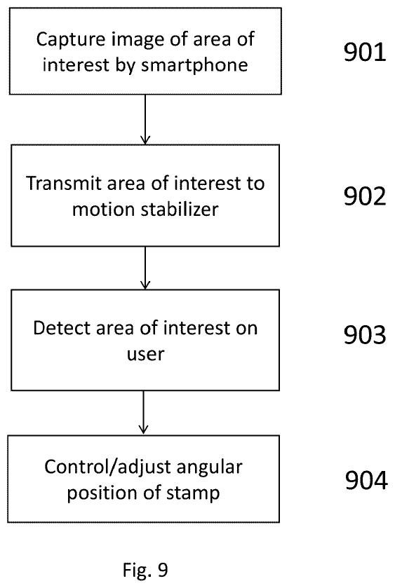

FIG. 9 shows a method performed according to the system of FIG. 8. In Step 901, the user's smartphone device captures an image of an area of interest on the user's face. The user may perform an operation on the image displayed on the smartphone to select the area of interest since it is expected that an image of the entire users' face is capture. In Step 902, the smartphone transmits the information of the selected area of interest to the motion stabilizer 150. In step 903, the embedded camera system and proximity sensor on the robotic stamp in combination with the circuitry of the motion stabilizer, detects the area of interest of the user when it enters a field of view of the camera system. In step 904, the robotic stamp is controlled to adjust its angular position to aim toward the area of interest as the user brings the device closer to the area of interest.

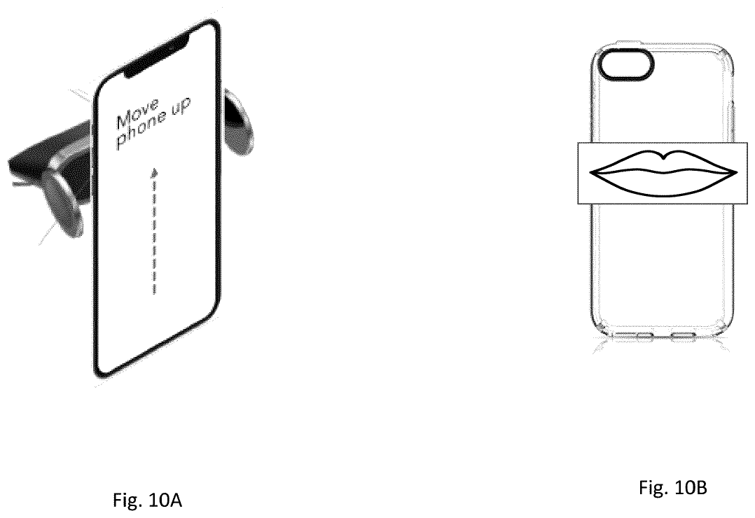

While the above embodiment depicts the robotic stamp as attached to the motion stabilizer, FIGS. 10A and 10B shown an embodiment where the robotic stamp 500 is mounted to a user's smartphone directly. As shown in FIG. 10A, in one embodiment, the clips 530 shown in FIGS. 5B and 5C above, may be used to mount the robotic stamp to the smartphone. Such mounting may be done by magnetic attachment. Alternatively, the clips 5B and 5C may be foldable with hinges and may move to allow the phone to fit into a recess on each of the clips.

FIG. 10B shows an additional embodiment where the robotic stamp is able to attached to a specialized phone case for the smartphone.

In the embodiment of FIG. 10, the user brings the smartphone with the stamp towards the area of interest and the smartphone guides the user with audible commands and visual commands in real time. The gimbal device on the stamp can be controlled wirelessly by the smartphone to adjust the angular position of the stamp as the user brings the stamp toward their face. Since the screen of the smartphone is turned away from the user, the phone can display text in mirror image form so that the user can face a mirror and see the text correctly on the phone through the mirror.

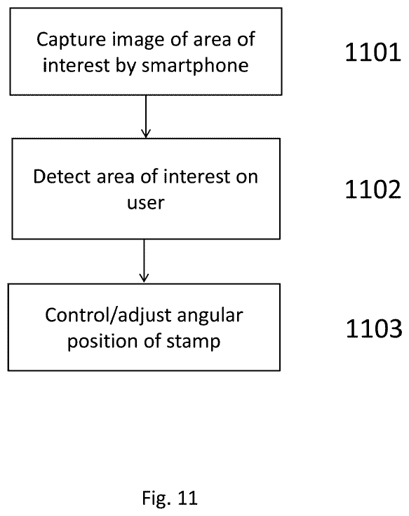

FIG. 11 shows a method performed according to the system of FIG. 10. In Step 1101, the user's smartphone device captures an image of an area of interest on the user's face. The user may perform an operation on the image displayed on the smartphone to select the area of interest since it is expected that an image of the entire users' face is capture. In Step 1102, the embedded camera system and proximity sensor on the robotic stamp in combination with the circuitry of the smartphone, detects the area of interest of the user when it enters a field of view of the camera system and/or the user's smartphone. In step 1104, the robotic stamp is controlled to adjust its angular position to aim toward the area of interest as the user brings the device closer to the area of interest.

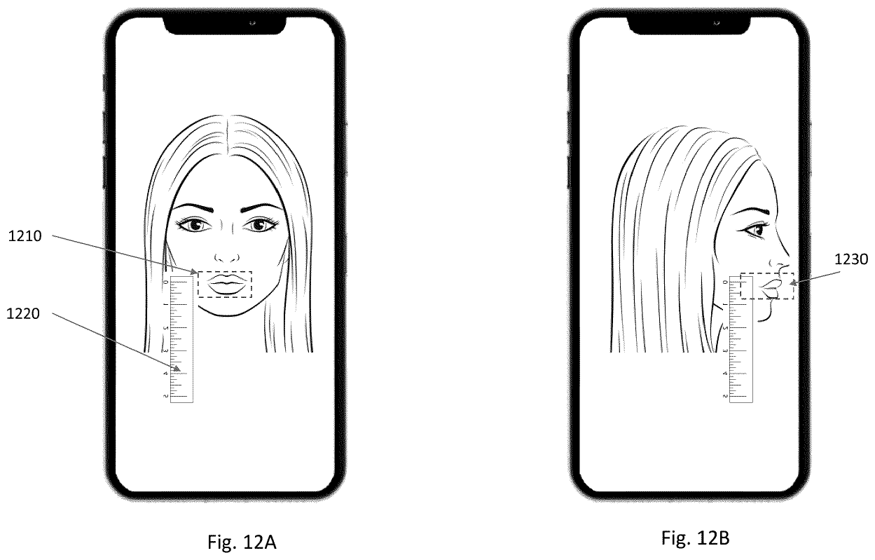

FIG. 12 shows how the stamp can be made to be personalized for the user based on the user's morphology and drawing objective. As shown in FIGS. 12A and 12B, within a specific application on a mobile user device, the user may take a front view “selfie” photo and a profile view photo. The application will capture dimensions of a target feature, such as a front lip region 1210 and a profile lip region 1230. For size guidance, the user may be asked to hold a ruler 1220 close to the target region for each photo. With these two images, the application will approximate the size and dimensions of the stamp to match the user's features.

As mentioned above, the stamp may be made out of the lipstick itself. Additionally, it can be bought “pre-loaded” with user's desired shade of lipstick. When manufacturing the stamp, or loading it with the lipstick, a uniform layer of lipstick may be loaded, or different shades could be loaded depending on the user's tastes.

Furthermore, with the above system, the embedded camera system can be used to apply different amounts of pressure with the stamp to apply thicker shades to different regions on the user's lips.

Obviously, numerous modifications and variations of the present invention are possible in light of the above teachings. It is therefore to be understood that within the scope of the appended claims, the invention may be practiced otherwise than as specifically described herein.

1. A stamp configured to apply a cosmetic formulation to a user, comprising:
a recessed area configured to receive the cosmetic formulation, the recessed area having a shape confirming to a target area of the user for applying the cosmetic formulation;
a connector configured to attach to an external device;
and processing circuitry configured to control an angular position of the stamp as the stamp is moved toward the target area of the user.

2. The stamp according to claim 1, wherein the cosmetic formulation is lipstick and the recessed area has a shape confirming to a user's lip region.

3. The stamp according to claim 1, further comprising an embedded camera and proximity sensor that are configured to detect the target area of the user as the stamp is moved toward the target area of the user.

4. The stamp according to claim 1, wherein further comprising a 2-axis gimbal motor system that is controlled by the processing circuitry to control the angular position of the stamp.

5. The stamp according to claim 1, further comprising a mounting mechanism configured to attach the stamp to a mobile user device.

6. The stamp according to claim 1, further comprising a connector configured to attach and communicatively couple the stamp to a motion stabilizing device.

7. A system comprising:
a stamp configured to apply a cosmetic formulation to a user, the stamp including:
a recessed area configured to receive the cosmetic formulation, the recessed area having a shape confirming to a target area of the user for applying the cosmetic formulation, and
a connector configured to attach to a motion stabilizing device; and
the motion stabilizing device configured to receive information of the target area of the user and control an angular position of the stamp as the stamp is moved toward the target area of the user.
